(19) United States
(12) Patent Application Publication
(10) Pub. No.: US 2026/0115125 A1
(43) Pub. Date: Apr. 30, 2026

(54) MASCARA COMPOSITION

(71) Applicant: L’ORÉAL, Paris (FR)

(72) Inventors: Huong BACH, Livingston, NJ (US); Laura MARTONE, Wall, NJ (US)

(73) Assignee: L’ORÉAL, Paris (FR)

(21) Appl. No.: 18/933,238

(22) Filed: Oct. 31, 2024

(51) Int. Cl. A61K 8/86; A61K 8/81; A61Q 1/10

(52) U.S. Cl. CPC A61K 8/86; A61K 8/8182; A61Q 1/10; A61K 2800/30

(57) ABSTRACT

Mascara compositions with strong, long last lift are provided. Such compositions generally include a water-soluble wax and a film former. The water-soluble wax includes a polyethylene glycol ether of cetearyl alcohol (such as Ceteareth-25). The film former includes a copolymer of n-vinyl pyrrolidone, methacrylamide, and n-vinylimidazole. The water-soluble wax may be present in an amount of 15% to 30% by weight of the composition. The film former may be present in an amount of 3% to 10% by weight of the composition. The compositions may include additional materials, such as a polyol, a chelating agent, a preservative, a skin active agent (such as an antioxidant). The composition may include water in an amount of 50% to 80% by weight of the composition. The composition may be substantially free of C2-C5 monoalcohols. The composition may be substantially free of a colorant.

### TECHNICAL FIELD

The present disclosure is drawn to cosmetic compositions, such as mascara compositions.

### BACKGROUND

Some mascara products are makeup products that generally appear as a transparent to translucent gel, including clear mascaras or tinted mascaras. Many such products are designed to be applied to both eyelashes and eyebrows. To date, such products do not provide high degree of lift and a strong hold when applied to lashes.

### BRIEF SUMMARY

In various aspects, a mascara composition may be provided. The mascara composition may include a water-soluble wax and a film former. In particular, the water-soluble wax may be a polyethylene glycol ether of cetearyl alcohol (such as Ceteareth-25). The water-soluble wax may consist of Ceteareth-25. The polyethylene glycol ether of cetearyl alcohol may be present in an amount of 15% to 30% by weight of the mascara composition. The film former may be a copolymer of n-vinyl pyrrolidone (VP), methacrylamide, and n-vinylimidazole. The copolymer of n-vinyl pyrrolidone, methacrylamide, and n-vinylimidazole may be present in an amount of 3% to 10% by weight of the mascara composition. The film former may consist of the copolymer of n-vinyl pyrrolidone, methacrylamide, and n-vinylimidazole.

The mascara composition may include a polyol in a total amount of 2% to 10% by weight of the mascara composition. The mascara composition may include a preservative. The mascara composition may include water in a total amount of 50% to 80% by weight of the mascara composition.

The mascara composition may include: (i) the water-soluble wax; (ii) the film former; (iii) the polyol; (iv) the preservative; (v) the water; and (vi) an antioxidant, a vitamin, and/or a chelating agent, and may be substantially free (or free) of all other materials.

The mascara composition may be substantially free of C2-C5 monoalcohols. The mascara composition may be substantially free of colorants. Aside from the water-soluble wax, the mascara composition may be substantially free of additional thickening agents.

In certain aspects, a method for providing improved mascara aesthetics may be provided. The method may include applying a mascara composition as disclosed herein to one or more lashes. The method may include allowing the mascara composition to remain on the one or more lashes for a period of time of, e.g., 1 hour to 48 hours.

### BRIEF DESCRIPTION OF FIGURES

FIG. 1 is a schematic illustration of a test for eyelash curl.

### DETAILED DESCRIPTION

As used herein, the terms “a,” “an,” and “the” are understood to encompass the plural as well as the singular.

As used herein, the term “free” or “completely free (of a component)” refers to compositions that do not contain the component in any measurable degree by standard means. As used herein, the term “substantially free (of a component)” refers to compositions that contain no appreciable amount of the component, for example, no more than about 1% by weight, or no more than about 0.5% by weight, or no more than about 0.3% by weight, such as no more than about 0.1% by weight, based on the weight of the composition.

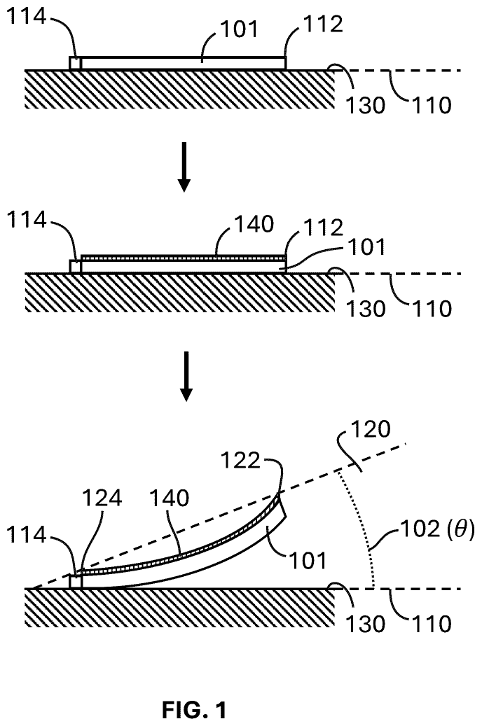

Disclosed are mascara compositions, and preferably clear mascara composition, configured to deliver a high degree of lift to lashes that with a strong hold that persists over time. This can be visualized referring to FIG. 1, where a set of bare fake lashes (101) may placed on a surface (130). One end (114) of the lashes may be used to affix the lashes to the surface (130). The angle of formed the lash at the point of attachment to a tip (112) of the bare lash prior to application of a mascara composition may be measured, relative to the surface (e.g., using a protractor). Here, the angle is zero (the lashes are parallel to surface, and for simplicity is not shown in FIG. 1. Then, a mascara composition (140) may be applied to the lashes (e.g., a brush may be used to apply the mascara composition, using multiple strokes). The composition may be applied from only one side of the lashes. After application, the curl angle (θ) (102) may be measured over time. As shown in FIG. 1, the curl angle (θ) (102) may generally be considered an angle formed between a first line (110) that is coplanar with the surface (130) to which the lash is attached, and a second line (120) that passes from a tip (122) of the lash (101) with the mascara (140) applied through a point (124) at which the lash (101) is affixed to the surface (130) and intersects the first line (110).

A “high” degree of lift may be considered to lift that provides a curl angle (θ) (102) of at least 15°, preferably at least 17.5°, more preferably at least 20°, and still more preferably at least 22.5°.

The degree of lift may be monitored over time. A mascara that provides a “strong” hold may be one in which the curl angle does not change (either increasing or decreasing) by more than 1° over a fixed period of time, such as 1 hour, 2 hours, 4 hours, 6 hours, or 8 hours.

Specifically, the disclosed mascara compositions are configured to utilize a synergy between a water-soluble wax and a strong film former to provide the structure to the gel as well as the strong lift and hold to the lashes.

### Water-Soluble Wax.

The disclosed mascara composition may include a water-soluble wax. As used herein, the term “wax” generally refers to a compound (such as a lipophilic or a hydrophilic compound, etc.) that is solid at room temperature (25° C.), with a solid/liquid reversible change of state, having a melting point of greater than or equal to 30° C., which may be up to 200° C. and in particular up to 120° C.

The water-soluble wax may be a polyethylene glycol ether of a fatty alcohol. The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fattyalcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

The water-soluble wax is preferably a polyethylene glycol ether of cetearyl alcohol, and most preferably ceteareth-25.

The water-soluble wax may have 20 to 30 ethylene oxide units The water-soluble wax may have 20, 21, 22, 23, 24, or 25 up to 25, 26, 27, 28, 29, or 30 ethylene oxide units, including all subranges and combinations thereof.

In some embodiments, the water-soluble wax may include ceteareth-25 and at least one additional water-soluble wax. In some embodiments, the water-soluble wax may include ceteareth-25 and may be free, or substantially free, of any additional water-soluble wax.

The polyethylene glycol ether of cetearyl alcohol may be present in a total amount of 15% to 30% by weight of the mascara composition. The polyethylene glycol ether of cetearyl alcohol may be present in a total amount of 15%, 16%, 17%, 18%, 19%, 20%, 21%, 22%, 23%, 24%, or 25%, up to 25%, 26%, 27%, 28%, 29%, or 30% by weight of the composition, including all ranges and subranges thereof.

The mascara composition may include ceteareth-25 in an amount of 15% to 30% by weight of the mascara composition. The ceteareth-25 may be present in an amount of 20%, 21%, 22%, 23%, 24%, or 25%, up to 25%, 26%, 27%, 28%, 29%, or 30% by weight of the composition, including all subranges and combinations thereof.

### Film Former.

The mascara composition may include a film former. The film former may be a copolymer of two or more vinyl monomers and an acrylamide monomer. The film former may be a copolymer of n-vinyl pyrrolidone (VP), methacrylamide, and n-vinylimidazole (INCI Name: VP/Methacrylamide/Vinyl imidazole copolymer).

In some embodiments, the film former may include the copolymer of n-vinyl pyrrolidone (VP), methacrylamide, and n-vinylimidazole, and at least one additional film former. In some embodiments, the film former may include the copolymer of n-vinyl pyrrolidone (VP), methacrylamide, and n-vinylimidazole, and may be free, or substantially free, of any additional film former.

If an additional film former is utilized, non-limiting examples of such film-forming polymers include keratin derivatives, such as keratin hydrolysates and sulphonic keratins; anionic, cationic, amphoteric or nonionic derivatives of chitin or chitosan; cellulose derivatives such as hydroxyethylcellulose, hydropropylcellulose, methylcellulose, ethylhydroxyethylcellulose, carboxymethylcellulose, and quaternized derivatives of cellulose; acrylic polymers or copolymers, such as polyacrylates or polymethacrylates (including homo- and co-polymers thereof), such as sodium polymethacrylate; polyvinylpyrrolidones (PVP) and vinyl copolymers, such as vinylpyrrolidone (VP)/vinyl acetate (VA) copolymer, allyl stearate/VA copolymer, and VP/eicosene copolymer, methyl vinyl ether-maleic anhydride copolymers, or vinyl acetate-crotonic acid copolymer; polyester, such as adipic acid/diethylene glycol/glycerin crosspolymer; water-dispersible anionic polyesteramide and/or polyester polymers comprising monomers bearing a functional group-SO₃M, in which M represents a hydrogen atom, an ammonium ion NH₄⁺ or a metal ion, such as, for example, an Na⁺, Mg²⁺, Ca²⁺, Cu²⁺, Fe²⁺ or Fe³⁺ ion.

In one embodiment, the film former consists of VP/Methacrylamide/Vinyl imidazole copolymer and VP/VA copolymer; in a preferred embodiment, the film former consists of VP/Methacrylamide/Vinyl imidazole copolymer.

The film former may be present in a total amount of 3% to 10% by weight of the mascara composition. The film former may be present in a total amount of 3%, 4%, or 5%, up to 5% 6%, 7%, 8%, 9%, or 10% by weight of the composition.

The copolymer of n-vinyl pyrrolidone, methacrylamide, and n-vinylimidazole may be present in an amount of 3% to 10% by weight of the mascara composition, and preferably 3% to 7% by weight of the composition. The copolymer of n-vinyl pyrrolidone, methacrylamide, and n-vinylimidazole may be present in a total amount of 3%, 4%, or 5%, up to 5% 6%, 7%, 8%, 9%, or 10% by weight of the composition, including all subranges and combinations thereof.

### Solvents.

The mascara composition may include one or more solvents. The solvents may include one or more volatile solvents. The term “volatile solvent” means a solvent that is capable of evaporating on contact with keratin materials in less than one hour, at room temperature and atmospheric pressure (760 mmHg). Such volatile solvents are typically liquid at room temperature, with a non-zero vapor pressure at room temperature and atmospheric pressure, ranging in particular from 0.13 Pa to 40 000 Pa (10⁻³ to 300 mmHg), in particular ranging from 1.3 Pa to 13,000 Pa (0.01 to 100 mmHg), and more particularly ranging from 1.3 Pa to 1300 Pa (0.01 to 10 mmHg). By way of example, the vapor pressure may be measured according to the static method or via the effusion method by isothermal gravimetry, depending on the vapor pressure (standard OCDE 104). The solvents may include one or more non-volatile solvents.

### Water.

The solvents may include water. The mascara composition may include water in an amount of, e.g., 50% to 80% by weight of the mascara composition. The mascara composition may include water in an amount of 50%, 55%, 60%, or 65%, up to 70%, 75%, or 80% by weight of the composition, including all subranges and combinations thereof. In a preferred embodiment, water is present in an amount of 55%-75%, and more preferably 60%-70% by weight of the composition.

In some embodiments, the solvent system may include only water and water-soluble materials. The solvent system may be free, or substantially free, of an oil. The term “oil” is intended to mean a non-aqueous compound, non-miscible in water, liquid, at 25° C. and atmospheric pressure (760 mmHg; 1.013.10⁵ Pa).

The solvent system may be free, or substantially free, of hydrocarbon-based oils. The term “hydrocarbon-based oil” refers to an oil mainly containing carbon and hydrogen atoms and possibly one or more functions chosen from hydroxyl, ester, ether and carboxylic functions. Such hydrocarbon-based oils may, e.g., contain from 8 to 16 carbon atoms, including linear and branched C8-C16 alkanes, such as C8-C16 isoalkanes of petroleum origin (also known as isoparaffins), such as isodecane, undecane, isododecane, tridecane, and/or isohexadecane.

The solvent system may be free, or substantially free, of silicone-based oils. The term “silicone oil” means an oil comprising at least one silicon atom, and notably at least one Si—O group, and more particularly an organopolysiloxane. Such silicone oils may be, e.g., dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyl-octyltrisiloxane, hexamethyldisiloxane, octamethyltrisiloxane, decamethyltetrasiloxane and dodecamethylpentasiloxane, or mixtures thereof.

### Polyols.

The solvent system may include one or more polyols. In some embodiments, the solvent system may include a plurality of polyols.

Non-limiting examples of polyols include ethylene glycol, propylene glycol, butylene glycol, hexylene glycol, pentylene glycol, diethylene glycol, dipropylene glycol, caprylyl glycol, 1,3 propanediol, glycerin, diglycerin, polyethylene glycols, or a mixture thereof.

In some preferred embodiments, the composition may include glycerin and be substantially free of all other polyols.

The mascara composition may include polyol(s) in a total amount of 2% to 10% by weight of the mascara composition. The mascara composition may include polyol(s) in a total amount of 1%, 2%, or 3%, up to 4%, 5%, 6%, 7%, 8%, 9%, or 10% by weight of the mascara composition, including all subranges and combinations thereof. In some preferred embodiments, the polyol(s) may be present in a total amount that is 6%-10% by weight of the composition. In some e preferred embodiments, the polyol(s) may be present in a total amount that is 2%-6% by weight of the composition.

The mascara composition may include a single polyol (such a single triol or a single diol) in a total amount of 2% to 6% by weight of the mascara composition, and be free or substantially free of all other polyols. The mascara composition may include a single triol in a total amount of 1%, 2%, or 3%, up to 4%, 5%, or 6% by weight of the mascara composition, including all subranges and combinations thereof, and be free or substantially free of all other polyols.

### Preservative.

The mascara composition may include a preservative, which may be present in any appropriate amount.

Preferred preservatives include hydroxyacetophenone, phenoxyethanol and mixtures thereof.

The preservative(s) may be present in a total amount of 0.01% to 3%. The preservative(s) may be present in a total amount of, e.g., 0.01%, 0.1%, 0.25%, or 0.5%, up to 1%, 2%, or 3% by weight of the composition, including all subranges and combinations thereof.

### Additional Components.

The mascara composition may optionally include one or more additional components.

### Skin Active Agent.

The mascara composition may include a skin active agent. The skin active agents(s) may be present in any appropriate amount. The skin active agents(s) may be present in a total amount of 0.001% to 5%. The skin active agents(s) may be present in a total amount of, e.g., 0.001%, 0.01%, 0.1%, 0.25%, or 0.5%, up to 0.5%, 1%, 2%, 3%, 4%, or 5% by weight of the composition, including all subranges and combinations thereof. The mascara composition may be free, or substantially free, of a skin active agent.

The skin active agent may include one or more antioxidants. The antioxidant(s) may include vitamins such as vitamin A (retinol), vitamin E (tocopherol) and other antioxidants such as extract of rosemary, vitamin B5 (panthenol), vitamin B3 (niacinamide), derivatives of these vitamins, mixtures thereof. The term “derivative” refers to compounds obtained by chemical modification of the mentioned compounds, and typically refer to esters, amides or ethers of said compounds. For example, a vitamin E derivative may include tocopheryl acetate. The antioxidants may include a carotenoid.

Preferably, the antioxidant(s) may include heterocyclic carboxylic acid compounds, amino acid compounds and hydroxylated benzene compounds optionally substituted on the phenyl ring by one or more (C1-C4) alkyl groups, in particular chosen from benzene alcohols preferably hydroxyacetophenones.

The antioxidant(s) may be present in any appropriate amount. The antioxidant(s) may be present in a total amount of 0.001% to 5%. The antioxidant(s) may be present in a total amount of, e.g., 0.001%, 0.01%, 0.1%, 0.25%, or 0.5%, up to 0.5%, 1%, 2%, 3%, 4%, or 5% by weight of the composition, including all subranges and combinations thereof. The mascara composition may be free, or substantially free, of an antioxidant.

The skin active agent may include one or more biological agents. The biological agent(s) may include amino acids, proteins, enzymes, and coenzymes. The biological agent(s) may include a glycosaminoglycan (such as hyaluronic acid)

The biological agent(s) may be present in any appropriate amount. The biological agent(s) may be present in a total amount of 0.001% to 5%. The biological agent(s) may be present in a total amount of, e.g., 0.001%, 0.01%, 0.1%, 0.25%, or 0.5%, up to 0.5%, 1%, 2%, 3%, 4%, or 5% by weight of the composition, including all subranges and combinations thereof. The mascara composition may be free, or substantially free, of a biological agent.

### Chelating Agents.

The mascara composition may include one or more chelating agents, such as a water-soluble chelating agent.

The at least one water soluble chelating agent may be an alkali metal hydroxyl polycarboxylate represented by an alkane containing from 1 to 4 carbon atoms, preferably containing 2 or 3 carbon atoms, substituted by 1, 2, or 3 hydroxyl groups (—OH), preferably by one (1) hydroxyl group, and further substituted by 2, 3, 4 or 5 carboxylate groups (—COOM), preferably by 2 or 3 carboxylate groups (—COOM), wherein the multiple groups M independently represent H or alkali metal, with the proviso that at least one of the groups M represents alkali metal, such as Na, K or Li, preferably all groups M represent alkali metal, such as Na, K or Li, preferably Na. More specifically, the at least one alkali metal hydroxyl polycarboxylate may be chosen from sodium tartrates, sodium citrates, potassium tartrates, potassium citrates, and hydrates thereof, preferably sodium citrates, in particularly trisodium citrate. Herein, sodium citrates are used to indicate monosodium citrate, disodium citrate and trisodium citrate, and other alkali metal hydroxyl polycarboxylates may be understood in a similar way.

Preferable chelating agents may comprise sodium citrate, tetrasodium ethylenediamine tetraacetate (EDTA), tetrasodium glutamate diacetate (GLDA), trisodium ethylenediamine disuccinate (EDDS), sodium phytate, or a mixture thereof.

The chelating agent may be present in any appropriate amount. The chelating agent(s) may be present in a total amount of 0.01% to 5%. The chelating agent(s) may be present in a total amount of, e.g., 0.01%, 0.1%, 0.25%, or 0.5%, up to 1%, 2%, 3%, 4%, or 5% by weight of the composition, including all subranges and combinations thereof. The mascara composition may be free, or substantially free, of a chelating agent.

The mascara composition may include: (i) the water-soluble wax; (ii) the film former; (iii) the polyol; (iv) the preservative; (v) the water; and (vi) an antioxidant, a vitamin, and/or a chelating agent, and may be substantially free, or free, of all other materials.

In some embodiments, the mascara composition may include: (i) the water-soluble wax; (ii) the film former; (iii) the polyol; (iv) the preservative; (v) the water; (vi) an antioxidant, a vitamin, and/or a chelating agent; (vii) optionally a colorant; (viii) optionally a C2-C5 monoalcohol; (ix) optionally an additional wax, and may be substantially free, or free, of all other materials.

### Additional Waxes.

While the mascara composition may include one or more additional waxes, (i.e., waxes in addition to the water-soluble wax disclosed herein), in preferred embodiments, the mascara composition is free, or substantially free, of additional waxes.

If present, the at least one additional wax may include a soft wax. As used herein, the term “soft wax” refers to waxes which have a melting point of no more than about 70° C., and preferably, a melting point of no more than about 60° C.

As used herein, the term “about [a number]” generally refers to values, as appropriate, that are within ±10%, ±8%, ±6%, ±5%, ±4%, ±3%, ±2%, ±1%, or ±0.5% of the number, or just including the number itself.

The at least one additional wax may be substantially free of a soft wax. The at least one additional wax may be free of a soft wax.

Non-limiting examples of soft waxes include paraffin wax, ozokerite, synthetic beeswax, beeswax, candelilla wax, hydrogenated jojoba wax, palm butter, sumac wax, polyglyceryl beeswax, siliconyl beeswax, ceresin wax, orange peel wax, silicone resin wax, alkyl dimethicone wax, Berry wax, koster wax, siliconyl candelilla wax, and montan wax.

If present, the at least one additional wax may include a hard wax. As used herein, the term “hard wax” refers to waxes other than soft waxes—that is, waxes which have a melting point greater than about 70° C., and preferably, a melting point greater than about 60° C. The at least one additional wax may be substantially free of a hard wax. The at least one additional wax may be free of a hard wax.

Non-limiting examples of hard waxes include carnauba wax, microcrystalline wax, polyethylene wax, hydrogenated castor oil, wax AC 540, hydrogenated castor wax, wax AC 400, rice bran wax, Alcohol polyethylene wax, sunflower seed wax, fischer-tropsch wax, Chinese insect wax, and shellac wax.

In some embodiments, the additional waxes may include at least one hard wax and at least one soft wax. In some embodiments, the additional waxes may include one hard wax one soft wax.

In various embodiments, additional waxes may be present in an amount of at least 0.001%, 0.01%, 0.1%, 0.5%, or 1%, up to 1.5%, 2%, 2.5%, 5%, or 10% by weight of the composition, including all subranges and combinations thereof.

### Additional Rheology Modifiers.

While the mascara composition may include one or more additional rheology modifiers (i.e., materials in addition to the water-soluble wax disclosed herein), in preferred embodiments, the mascara composition is free, or substantially free, of additional rheology modifiers. In some embodiments, the mascara composition may be free, or substantially free, of hydrophilic rheology modifiers. In some embodiments, the mascara composition may be free, or substantially free, of lipophilic rheology modifiers (such as hectorites, etc.).

Non-limiting examples of rheology modifiers (sometimes referred to as thickening agents) include, e.g., xanthan gum, guar gum, biosaccharide gum, cellulose, acacia Seneca gum, sclerotium gum, agarose, pechtin, gellan gum, hyaluronic acid, ammonium polyacryloyldimethyl taurate, ammonium acryloyldimethyltaurate/VP copolymer, sodium polyacrylate, acrylates copolymers, polyacrylamide, carbomer, and/or acrylates/C10-30 alkyl acrylate crosspolymer.

### C2-C5 Monoalcohols

While the mascara composition may include one or more C2-C5 monoalcohols, the mascara composition is preferably free, or substantially free, of of C2-C5 monoalcohols. Such C2-C5 monoalcohols include, e.g., ethanol, propanol, butanol, isopropanol, etc. As used herein, the term “alcohol” generally refers to an organic compound having at least one hydroxyl (—OH) functional group bound to a saturated carbon atom. “Monoalcohol” therefore refers to an alcohol having only a single hydroxyl group bound to a saturated carbon atom.

In some embodiments, the mascara composition may be free of any non-phenyl monoalcohol. For example, the composition may include, e.g., phenoxyethanol (a phenyl-based ether monoalcohol) and phenylpropanol (a phenyl-based monoalcohol), and may be free or substantially free of all other monoalcohols.

In various embodiments, the C2-C5 monoalcohols may be present in any appropriate amount, such as at least 0.001%, 0.01%, 0.1%, 0.5%, or 1%, up to 1.5%, 2%, 2.5%, 5%, 10%, or 20% by weight of the composition, including all subranges and combinations thereof.

### Colorants.

While the mascara composition may include one or more colorants, the mascara composition is preferably free, or substantially free, of colorants. For example, sometimes colorants may be added to give a specific color (e.g., a functional use of colorant) in which load might be relatively high (e.g., 5% or more, 10% or more, 15% or more, or 20% or more) as functional use of it). In other examples, colorants may be added more for non-functional purposes, such as to hide white cast of the remainder of the formulation and/or to give small amount of tint to the product; in such cases, the load may be relatively low (e.g., 10% or less, 5% or less, 4% or less, 3% or less, 2% or less, or 1% or less).

The colorant may be, e.g., a pigment. Examples of suitable pigments include, but are not limited to, inorganic pigments, organic pigments, lakes, pearlescent pigments, iridescent or optically variable pigments, and mixtures thereof. The pigments may be chosen from the organic and/or mineral pigments known in the art. The pigments may be in the form of powder or of pigmentary paste. They may be coated or uncoated. Said pigments may optionally be surface treated within the scope of the present invention but are not limited to treatments such as silicones, perfluorinated compounds, lecithin, and amino acids.

The pigments may be chosen, for example, from inorganic pigments, organic pigments, lakes, pigments with special effects such as nacres or glitter flakes, and mixtures thereof. Non-limiting examples of inorganic pigments include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The pigment may be an inorganic pigment. Non-limiting examples include iron oxides, chromium oxides, manganese violet, ultramarine blue, chromium hydrate, ferric blue and titanium oxide.

The pigment may be an organic pigment. Non-limiting examples include nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

In particular, the white or colored organic pigments may be chosen from carmine, carbon black, aniline black, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and pigments obtained by oxidative polymerization of indole or phenolic derivatives. Non-limiting examples of organic pigments and lakes include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

The pigments in accordance with the invention may also be in the form of composite pigments. These composite pigments may be compounds especially of particles comprising a inorganic core, at least one binder for ensuring the binding of the organic pigments to the core, and at least one organic pigment at least partially covering the core.

The organic pigment may also be a lake. The term “lake” refers dyes adsorbed onto insoluble particles, the assembly thus obtained remaining insoluble during use.

The inorganic substrates onto which the dyes are adsorbed are, for example, alumina, silica, calcium sodium borosilicate or calcium aluminum borosilicate, and aluminum.

Among the dyes, mention may be made of cochineal carmine. Non-limiting examples of dyes include: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090). An example of a lake is D&C Red 7 (CI 15 850:1).

The pigment may also be a pigment with special effects. The term “pigments with special effects” refers pigments that generally create a non-uniform colored appearance (characterized by a certain shade, a certain vivacity and a certain lightness) that changes as a function of the conditions of observation (light, temperature, observation angles, etc.). They thus contrast with colored pigments that afford a standard uniform opaque, semi-transparent or transparent shade.

Several types of pigment with special effects exist: those with a low refractive index, such as fluorescent, photochromic or thermochromic pigments, and those with a higher refractive index, such as nacres or glitter flakes.

Examples of pigments with special effects that may be mentioned include nacreous pigments such as titanium mica coated with an iron oxide, mica coated with an iron oxide, mica coated with bismuth oxychloride, titanium mica coated with chromium oxide, titanium mica coated with an organic dye especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or tint.

Non-limiting examples of nacres include gold-colored nacres sold especially by the company Engelhard under the name Gold 222C (a CLOISONNÉ® pigment), Sparkle gold (a TIMICA® pigment), Gold 4504 (a CHROMA-LITE® pigment) and Monarch gold 233X (a CLOISONNÉ® pigment); the bronze nacres sold especially by the company Merck under the name Bronze fine (17384) (a COLORONA® pigment) and Bronze (17353) (a COLORONA® pigment), by the company Eckart under the name PRESTIGE™ Bronze pigment and by the company Engelhard under the name Super bronze (a CLOISONNÉ® pigment); the orange nacres sold especially by the company Engelhard under the name Orange 363C (a CLOISONNE® pigment) and Orange MCR 101 and by the company Merck under the name Passion orange (a COLORONA® pigment) and Matte orange (17449); the brown nacres sold especially by the company Engelhard under the name Nu-antique copper 340XB (a CLOISONNE® pigment) and Brown CL4509 (a CHROMA-LITE® pigment); the nacres with a copper tint sold especially by the company Engelhard under the name Copper 340A (a TIMICA® pigment) and by the company Eckart under the name Prestige Copper; the nacres with a red tint sold especially by the company Merck under the name Sienna fine (17386) (a COLORONA® pigment); the nacres with a yellow tint sold especially by the company Engelhard under the name Yellow (4502) (a CHROMA-LITE® pigment); the red nacres with a gold tint sold especially by the company Engelhard under the name Sunstone G012 (a GEMTONE® pigment); the black nacres with a gold tint sold especially by the company Engelhard under the name Nu antique bronze 240 AB (a TIMICA® pigment), the blue nacres sold especially by the company Merck under the name Matte blue (17433), Dark Blue (117324) (a COLORONA® pigment), the white nacres with a silvery tint sold especially by the company Merck under the name XIRONA® Silver pigment, and the golden-green pink-orange nacres sold especially by the company Merck under the name Indian summer (a XIRONA® pigment), and mixtures thereof.

In addition to nacres on a mica support, multilayer pigments based on synthetic substrates such as alumina, silica, sodium calcium borosilicate or calcium aluminum borosilicate, and aluminum, may be envisaged.

Non-limiting examples of pearlescent pigments include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

Mention may also be made of pigments with an interference effect that are not fixed onto a substrate, for instance liquid crystals or holographic interference glitter flakes. Pigments with special effects also comprise fluorescent pigments, whether these are substances that are fluorescent in daylight or that produce an ultraviolet fluorescence, phosphorescent pigments, photochromic pigments, thermochromic pigments and quantum dots.

The variety of pigments that may be used in the present invention makes it possible to obtain a wide range of colors, and also particular optical effects such as metallic effects or interference effects.

The effective diameter of the pigment particles may generally be between 10 nm and 200 μm, such as between 20 nm and 80 μm, such as between 30 nm and 50 μm.

In various embodiments, the colorants may be present in any appropriate amount, such as at least 0.001%, 0.01%, 0.1%, 0.5%, or 1%, up to 1.5%, 2%, 2.5%, 5%, 10%, 20%, or 30% by weight of the composition, including all subranges and combinations thereof. In some embodiments, the colorant may be present in an amount of 10% or less, preferably 5% or less, and more preferably 2.5% or less.

### Emulsifiers.

While the mascara composition may include one or more emulsifiers, the composition is preferrable free, or substantially free, of emulsifiers.

In some embodiments, the emulsifier may be a non-ionic emulsifier. Non-limiting examples of non-ionic emulsifiers include, e.g., saturated or unsaturated poly(oxyethylenated) C8-C30 monoesters or polyesters; saturated or unsaturated poly(oxyethylenated) C8-C30 alcohols; polyoxyethylenated or non-polyoxyethylenated, saturated or unsaturated C8-C30 monoesters or polyesters of sorbitan; polyoxyethylenated or non-polyoxyethylenated, saturated or unsaturated C8-C30 monoethers or polyethers of sorbitan; alkyl and polyalkyl glycosides or polyglycosides; saturated or unsaturated C8-C30 monoesters and polyesters of sucrose; polyoxyethylenated or non-polyoxyethylenated, saturated or unsaturated C8-C30 monoesters and polyesters of glycerol; polyoxyethylenated or non-polyoxyethylenated, saturated or unsaturated C8-C30 monoethers or polyethers of glycerol; and mixtures thereof.

To improve the lengthening effect of the disclosed mascara, the mascara composition may also include one or more fibers, such as a plurality of fibers. Such fibers may include, e.g., cellulose or cellulose-based fibers. In various embodiments, the fibers may be present in any appropriate amount, such as at least 0.001%, 0.01%, 0.1%, 0.5%, or 1%, up to 1.5%, 2%, 2.5%, 5%, or 10% by weight of the composition, including all subranges and combinations thereof. However, in some preferred embodiments, the mascara composition may be free, or substantially free, of fibers.

In various aspects, a method for providing improved mascara aesthetics may be provided. The method may include applying a mascara composition as disclosed herein to one or more lashes. The method may include allowing the mascara composition to remain on the one or more lashes for a period of time. The period of time may be, e.g., from 1 hour to 48 hours.

### Example

The following example formulations were produced using conventional manufacturing techniques. Here, the materials other than the film-former are added together and mixed until uniform. The main batch is then heated to 55° C. to 75° C. The film former is then introduced to the main batch Adding it below 55° C. will cause the batch to solidify, hindering proper incorporation. Conversely, exceeding 75° C. will subject the film former to excessive heat, negatively impacting the gel penetration and ultimately compromising the desired makeup result. Furthermore, achieving uniform distribution of the film former creates aeration, which, if done improperly (e.g., due to solidification at low temperatures), can also adversely affect the final makeup result.

Table 1 illustrates an exemplary formula (E1) that was produced, along with two commercially available clear mascara formulations.

TABLE 1

| | E1 | C1 | C2 |
|---|---|---|---|
| Water | q.s. | q.s. | q.s. |

Rheology Modifiers

| | | | |
|---|---|---|---|
| Ceteareth-25 | 25 | | |
| Beheneth-30 | — | | 20 |
| Other Thickeners | — | 1-2 | <1 |

Film Former

| | | | |
|---|---|---|---|
| VP/MAA/VI Copolymer | 4 | — | — |
| VP/VA Copolymer | — | — | 6 |

Polyols

| | | | |
|---|---|---|---|
| Glycerin | 3.5 | 3 | 1-1.5 |
| Other Polyols (Diols) | <1 | <1 | 7-8 |
| Ethanol | — | 5 | 3 |
| pH Adjuster | — | <1 | — |
| Chelating Agent | <1 | <1 | — |
| Preservative | <1 | <1 | <1 |
| Antioxidant | <1 | <1 | — |

Table 2 shows four comparative formulations (C3-C6) that were produced, that did not include a water-soluble wax.

TABLE 2

| | C3 | C4 | C5 | C6 |
|---|---|---|---|---|
| Water | q.s. | q.s. | q.s. | q.s. |
| Ceteareth-25 | — | — | — | — |
| Add'l Thickener | 2 | ~1.5 | 1 | <1 |

Film Former

| | | | | |
|---|---|---|---|---|
| VP/MAA/VI Copolymer | 18-19 | 4 | 4 | 5 |
| VP/VA Copolymer | — | 6 | 6 | — |

Polyols

| | | | | |
|---|---|---|---|---|
| Glycerin | — | 3.5 | 3.5 | 3.5 |
| Other Polyols (Diols) | <1 | <1 | <1 | <1 |
| Ethanol | 2 | 11 | 9 | 3 |
| Chelating Agent | — | <1 | <1 | <1 |
| Preservative | <1 | <1 | <1 | <1 |
| Antioxidant | <1 | <1 | <1 | <1 |
| Biological Agent | — | 1 | 1-1.5 | 1-1.5 |

Table 3 shows two comparative formulations (C7-C8), and three exemplary formulations (E2-E4). These formulas included the water-soluble wax. Comparative formula C8 did not include any film former.

TABLE 3

| | C7 | C8 | E2 | E3 | E4 |
|---|---|---|---|---|---|
| Water | q.s. | q.s. | q.s. | q.s. | q.s. |
| Ceteareth-25 | 15 | 25 | 20 | 25 | 25 |

Film Former

| | | | | | |
|---|---|---|---|---|---|
| VP/MAA/VI Copolymer | 4 | — | 4 | 4 | 8 |
| VP/VA Copolymer | 3 | — | 3 | 3 | — |

Polyols

| | | | | | |
|---|---|---|---|---|---|
| Glycerin | 3.5 | 3.5 | 3.5 | 3.5 | 3.5 |
| Other Polyols (Diols) | <1 | — | <1 | <1 | <1 |
| Ethanol | 3 | — | — | — | — |

| | |
|---|---|
| Chelating Agent | <1 |
| Preservative | <1 |
| Antioxidant | <1 |

The various formulas were then applied to fake lashes, which were visually evaluated after 30 strokes.

The formulas in Table 1 were evaluated on various factors, including curl angle. As described herein, bare lashes for each formula were applied to a surface, and a protractor is used to measure the curl angle of bare lashes. 30 strokes of each product were applied to their respective fake lashes. A photograph was taken at time t=0 hours. After an hour, the samples were completely dry, and a second photograph was taken at time t=1 hour. The photographs were sent to a computer, and the computer image processing tools were used to measure the curl angle. The results are shown in Table 4, below. As can be seen, the exemplary formula is markedly superior with respect to providing an improved curl angle over the comparative formulations.

TABLE 4

| | Curl Angle at t = 0 hours | Curl Angle at t = 1 hour |
|---|---|---|
| C1 | 9° | 7° |
| C2 | 13° | 14° |
| E1 | 24° | 24° |

The various formulas in Tables 2 and 3 were also evaluated on various factors. One evaluation related to considering thickness, tackiness, and structure of the mascara as they relate to the speed at which curl is formed on lashes and the “push” (e.g., how well it volumizes and lengthens lashes).

Here, for example, C3 was considered too thin, not providing enough grip for a quick life of the lashes. C4 and C5 were similarly too thin and did not have enough push during application. C6 was a thick gel, but did not have the structure to provide the needed good grip to create a desired curl or to provide sufficient push. C7 was too thin, while C8 was too thick (and had low deposit). E2-E4 all performed well, creating strong, long-lasting lift, with good push. E4 was near the upper limit of film former, however, as the lashes had begun to exhibit a reversed curl (e.g., the tips began to curl down, instead of up).

The structure of exemplary formula E1 was probed via a penetration test. As is known, a penetrometry measurement consists of determining the force of resistance which the product opposes during its penetration, under defined conditions in the specification of production (container, temperature, standardized measurement spindle, pre-speed, speed, trigger force, depth, point where the result of the force must be taken, etc.). Here, measurement is performed using a Texture Analyzer with software and a 5 kg force sensor, a compression trigger of 2 g, a pre-speed and measurement speed of 0.5 mm/s, and a depth of 5 mm. The temperature of the formula to be tested was 20° C.±2° C. A mobile of the Texture Analyzer (P0.5) is placed on the sensor. A force calibration is performed after putting on the mobile. The sample is then positioned in the center, a measurement is started, and a value of the force at the target distance is identified. The results are shown in Table 5 below.

TABLE 5

| | Penetration (g) |
|---|---|
| Competitive Product (A Hair Gel) | 573.8 |
| E1 (Lot 1) | 236.9 |
| E1 (Lot 2) | 172.7 |
| E1 (Lot 3) | 163.6 |
| E1 (Lot 4) | 161.6 |

Penetration is a measure of hardness, and as can be seen, exemplary formula E1 has structure, but substantially less than the competitive product. It is believed that having formulations that result in products having penetrations in the 100 g-300 g range is desirable, with a more preferable range being 100-170 g, providing enough structure for a stable formulation and desirable performance characteristics. Thus, modifications to the formula that may bring the penetration values outside this range are less desirable.

What is claimed is:

1. A mascara composition, comprising:
a water-soluble wax including a polyethylene glycol ether of cetearyl alcohol; and
a film former including a copolymer of n-vinyl pyrrolidone, methacrylamide, and n-vinylimidazole;

2. The mascara composition of claim 1, wherein the polyethylene glycol ether of cetearyl alcohol is present in an amount of 15% to 30% by weight of the mascara composition.

3. The mascara composition of claim 2, wherein the polyethylene glycol ether of cetearyl alcohol is Cetereath-25.

4. The mascara composition of claim 2, wherein the water-soluble wax consists of Cetereath-25.

5. The mascara composition of claim 1, wherein the copolymer of n-vinyl pyrrolidone, methacrylamide, and n-vinylimidazole is present in an amount of 3% to 10% by weight of the mascara composition.

6. The mascara composition of claim 5, wherein the film former consists of the copolymer of n-vinyl pyrrolidone, methacrylamide, and n-vinylimidazole.

7. The mascara composition of claim 1, further comprising a polyol in a total amount of 2% to 10% by weight of the mascara composition.

8. The mascara composition of claim 7, further comprising a preservative.

9. The mascara composition of claim 8, further comprising water in a total amount of 50% to 80% by weight of the mascara composition.

10. The mascara composition of claim 9, wherein the mascara composition includes:
the water-soluble wax;
the film former;
the polyol;
the preservative;
the water; and
a skin active agent and/or a chelating agent;
wherein the mascara composition is substantially free of all other materials.

11. The mascara composition of claim 1, wherein the mascara composition is substantially free of C2-C5 monoalcohols.

12. The mascara composition of claim 1, wherein the mascara composition is substantially free of a colorant.

13. The mascara composition of claim 1, wherein the mascara composition is substantially free of additional waxes.

14. A method for providing improved lash lift and hold, comprising:
applying a mascara composition of claim 1 to one or more lashes; and
allowing the mascara composition to remain on the one or more lashes for a period of time, the period of time being from 1 hour to 48 hours.
